(19) United States
(12) Patent Application Publication
(10) Pub. No.: US 2021/0268037 A1
(43) Pub. Date: Sep. 2, 2021

(54) ANAEROSTIPES HADRUS FOR USE IN PROMOTING HEALTH

(71) Applicants: Second Genome, Inc., Brisbane, CA (US); University College Cork - National University of Ireland, Cork (IE)

(72) Inventors: Shoko Kawana, Brisbane, CA (US); Todd Z. DeSantis, Brisbane, CA (US); Marcus J. Claesson, Cork (IE); Feargal J. Ryan, Royal Park, SA (AU)

(21) Appl. No.: 17/255,126

(22) PCT Filed: Jun. 25, 2019
(86) PCT No.: PCT/US2019/039064

§ 371 Date: Dec. 22, 2020

Related U.S. Application Data
(60) Provisional application No. 62/689,278, filed on Jun. 25, 2018.

(51) Int. Cl. A61K 35/742; A61P 1/00

(52) U.S. Cl. CPC A61K 35/742; A61P 1/00

(57) ABSTRACT

The disclosure relates to live biotherapeutic products, probiotics, pharmaceutical compositions comprising said probiotics, and methods of using them to treat a gastrointestinal disease or disorder. In some aspects, the disclosure provides such compositions comprising strains of the bacterium Anaerostipes hadrus and their uses in treating intestinal diseases or disorders.

### CROSS-REFERENCE TO RELATED APPLICATIONS

This application claims priority to U.S. Provisional Application Ser. No. 62/689,278, filed on Jun. 25, 2018, the contents of which is hereby incorporated by reference in its entirety.

### CROSS-REFERENCE TO A SEQUENCE LISTING

The contents of the text file submitted electronically herewith are incorporated herein by reference in their entirety: A computer readable format copy of the Sequence Listing filename: SG301Dseqlist.txt (−13684 bytes on disc), created on Jun. 25, 2018.

### FIELD

The present disclosure relates to novel and therapeutically effective compositions comprising microbes, specifically Anaerostipes hadrus. The microbe compositions have application, inter alia, in the treatment, amelioration and/or prevention of gastrointestinal inflammatory diseases and/or epithelial barrier function disorders.

### BACKGROUND

Inflammatory bowel disease (IBD) is a heterogeneous disease of unknown etiology resulting in frequent and bloody bowel movements accompanied with histopathological damage to the gastrointestinal mucosa (Zhang et al., 2017, Front Immunol, 8:942). While specific triggers of disease remain poorly defined, one proposal of disease progression suggests a breakdown of intestinal barrier function allows bacteria or bacterial components to translocate into mucosal tissue (Coskun, 2014, Front Med (Lausanne), 1:24; Martini et al., 2017, Cell Mol Gastroenterol Hepatol, 4:33-46). Bacterial translocation results in activation of inflammatory signaling which triggers additional barrier disruption, resulting in a cyclic amplification loop of barrier disruption, bacterial translocation and inflammation. While many current therapies target inflammation, the lack of therapies promoting mucosal healing provides an opportunity for novel therapies promoting epithelial repair and intestinal barrier integrity.

The microbiome of the gastrointestinal tract comprises a diverse array of microorganisms, primarily prokaryotes, which play a significant role in the health of the host organism. The complexity of the microbiome, in terms of both its population makeup and composite function, has recently become an intense area of study as research increasingly shows that manipulation of the microbiome can provide health benefits and may be effective in treating a number of diseases and disorders. Currently, a number of probiotics are marketed which contain live bacteria and yeast and are believed to augment the benefits of these microbes which naturally occur in the human body. Increasingly, live biotherapeutic products (LBPs) are being developed for controlled clinical studies and regulatory approval in the treatment of disease.

### BRIEF SUMMARY OF THE DISCLOSURE

A study performed as described herein was designed to analyze bacteria present in the intestine of healthy subjects and in patients diagnosed with an inflammatory bowel disease. Importantly, this study was able to characterize both bacterial abundance and metabolic activity. Moreover, the data were correlated with host gene expression analysis conducted via RNAseq analysis. The results of this study and analysis identified Anaerostipes hadrus as being depleted in subjects diagnosed with ulcerative colitis or Crohn's disease, thereby showing that increased levels of A. hadrus in the intestine can be beneficial to the intestinal health of a subject.

In one aspect, a method for treating inflammatory bowel disease in a subject in need thereof is provided, comprising administering to the subject a composition comprising a therapeutically effective amount of a bacterium which has a 16S rRNA gene that is at least 75%, 80%, or 85% identical to SEQ ID NO:1, SEQ ID NO:2, SEQ ID NO:3, SEQ ID NO:4, SEQ ID NO:5, SEQ ID NO:6 or SEQ ID NO:7. The compositions as provided herein typically comprises a population of the bacterium, wherein each member of the population has a substantially identical 16S rRNA gene sequence.

In some embodiments, the bacterium has a 16S rRNA gene that is at least 90%, 91%, 92%, 93%, 94%, 95%, 96%, 97%, 98%, or 99%, 99.1%, 99.2%, 99.3%, 99.4%, 99.5%, 99.6%, 99.7%, 99.8%, 99.9%, or 100% identical to SEQ ID NO:1, SEQ ID NO:2, SEQ ID NO:3, SEQ ID NO:4, SEQ ID NO:5, SEQ ID NO:6 or SEQ ID NO:7.

In some embodiments, the bacterium is a strain of Anaerostipes hadrus (A. hadrus).

In some embodiments, the subject has been diagnosed with intestinal inflammation. In other embodiments, the intestinal inflammation is in the small intestine and/or the large intestine. In still other embodiments, the intestinal inflammation is in the rectum. In yet other embodiments, the patient has at least one symptom associated with inflammatory bowel disease.

In some embodiments, the subject has been diagnosed with and/or is suffering from an inflammatory bowel disease (IBD). In other embodiments, the IBD is selected from the group consisting of Crohn's disease (CD), ulcerative colitis (UC), pouchitis, irritable bowel syndrome, an enteric infection, GI mucositis, and a combination thereof. In some embodiments, the subject has been diagnosed with CD. In other embodiments, the subject has been diagnosed with UC.

In some embodiments, the subject has been diagnosed with an intestinal ulcer. In other embodiments, the patient has been diagnosed with draining enterocutaneous and/or rectovaginal fistulas.

In some embodiments, the subject has been diagnosed with CD. In other embodiments, the CD is mildly active CD. In still other embodiments, the CD is moderately to severely active CD. In yet other embodiments, the subject has been diagnosed with pediatric CD.

In some embodiments, the subject has been diagnosed with UC. In other embodiments, the UC is mildly active UC. In still other embodiments, the UC is moderately to severely active UC. In still other embodiments, the subject has been diagnosed with pediatric UC.

In some embodiments, the subject has been diagnosed with mucositis. In other embodiments, the mucositis is oral mucositis. In still other embodiments, the mucositis is chemotherapy-induced mucositis, radiation therapy-induced mucositis, chemotherapy-induced oral mucositis, or radiation therapy-induced oral mucositis. In yet other embodiments, the mucositis is gastrointestinal mucositis. In still other embodiments, the gastrointestinal mucositis is mucositis of the small intestine, the large intestine, or the rectum.

In some embodiments, the subject is in clinical remission from an IBD. In other embodiments, the subject is in clinical remission from UC, pediatric UC, CD, or pediatric CD.

In some embodiments, the subject has an inflammatory bowel disease or disorder other than CD or UC.

In some embodiments, the administering reduces gastrointestinal inflammation and/or reduces intestinal mucosa inflammation associated with inflammatory bowel disease in the subject. In other embodiments, the administering improves intestinal epithelial cell barrier function or integrity in the subject.

In some embodiments, after administering, the subject experiences a reduction in at least one symptom associated with an inflammatory bowel disease or disorder. In other embodiments, the at least one symptom is selected from the group consisting of abdominal pain, blood in stool, pus in stool, fever, weight loss, frequent diarrhea, fatigue, reduced appetite, nausea, cramps, anemia, tenesmus, and rectal bleeding. In still other embodiments, after administering, the subject experiences reduced frequency of diarrhea, reduced blood in stool and/or reduced rectal bleeding.

In some embodiments, the subject has experienced inadequate response to conventional therapy. In other embodiments, the conventional therapy is treatment with an aminosalicylate, a corticosteroid, a thiopurine, methotrexate, a JAK inhibitor, a sphingosine 1-phosphate (S1P) receptor inhibitor, an anti-integrin biologic, an anti-ILI2/23R or anti-IL23/pI0 biologic, and/or an anti-tumor necrosis factor agent or biologic.

In some embodiments, the administering increases the amount of mucin in the intestinal lumen of the subject.

In some embodiments, the administering comprises oral administering of the pharmaceutical composition to the subject. In other embodiments, the administering is to the gastrointestinal lumen.

In some embodiments, the subject is also administered at least one second therapeutic agent. In other embodiments, the at least one second therapeutic agent is selected from the group consisting of a prebiotic, an anti-diarrheal, an anti-inflammatory agent, an antibody, an antibiotic, or an immunosuppressant. In still other embodiments, the at least one second therapeutic agent is an aminosalicylate, a steroid, or a corticosteroid. In other embodiments, the at least one second therapeutic agent is selected from the group consisting of adalimumab, pegol, golimumab, infliximab, vedolizumab, ustekinumab, tofacitinib, and certolizumab or certolizumab pegol.

In one aspect, a composition comprising a therapeutically effective amount of a bacterium is provided, wherein the bacterium comprises a 16S rRNA gene that is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO:8. In some embodiments, the composition further comprises a therapeutically acceptable excipient. In some embodiments, the compositions as provided herein comprise a population of the bacterium, wherein each member of the population has a substantially identical 16S rRNA gene sequence.

In some embodiments, the bacterium was substantially purified from a bacterial culture. In other embodiments, the bacterium is viable.

In some embodiments, the bacterium is a strain of A. hadrus.

In another aspect, a method for diagnosing an IBD in a subject is provided, wherein the method comprises measuring A. hadrus in a sample from the subject.

In some embodiments, the sample is a fecal sample.

In some embodiments, the measuring of A. hadrus in a sample from the subject comprises detecting and quantitating genomic DNA and/or RNA specific to A. hadrus. In other embodiments, the genomic DNA is 16S DNA. In still other embodiments, the RNA is 16S RNA In yet other embodiments, the measuring comprises detecting and quantitating a sequence comprising SEQ ID NO:1, SEQ ID NO:2, SEQ ID NO:3, SEQ ID NO:4, SEQ ID NO:5, SEQ ID NO:6, SEQ ID NO:7 or fragment thereof.

In some aspects, a method of diagnosing a subject as having an IBD is provided wherein the method comprises detecting and/or quantitating A. hadrus in a sample from the subject.

In some embodiments, the sample is a fecal sample. In other embodiments, the sample is an intestinal biopsy.

In some embodiments, the method comprises detecting and/or quantitating in the sample a nucleic acid sequence that is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO:1, SEQ ID NO:2, SEQ ID NO:3, SEQ ID NO:4, SEQ ID NO:5, SEQ ID NO:6 or SEQ ID NO:7, or a fragment thereof.

### BRIEF DESCRIPTION OF THE FIGURES

FIGS. 1A and 1B. Sequence counts distribution at each step of sequencing data processing as described in Example 1. Shown are read counts (y-axis) categorized as raw counts, quality filtered, merged, and chimera removed for both DNA-16S (FIG. 1A) and RNA-16S (FIG. 1B).

FIGS. 2A and 2B. Schematics showing alpha diversity among CD, UC and control groups by Kruskal-Wallis test to assess both observed richness (left chart) and Shannon diversity (right chart). Analyses are shown for both DNA-16S (FIG. 2A) and RNA-16S (FIG. 2B).

FIG. 3. Schematic of the principal coordinate analysis (PCoA) using Bray-Curtis dissimilarity matrix. Circles denote DNA-16S and triangles RNA-16S samples. Same samples are connected by a line between a circle and triangle. Ellipses represent 80% of the samples belong to the group and are indicated on the schematic by arrows for CD, UC and control. The solid ellipse border indicates DNA-16S analysis; the dashed ellipse border indicates RNA-16S analysis.

FIG. 4. Schematic providing volcano plots to show dynamic microbiota RSVs shift between case (UC or CD) and controls. A, Volcano plots. Dynamic RSVs (adjusted p-value<0.05, absolute log 2 fold change>1, and non-zero sequence counts in over 75% of the subjects in at least one group) were shaded as shown in the legends.

FIG. 5. Schematic showing proportional abundance of A. hadrus in UC, CD and controls. Mean, 1st and 3rd quartiles are depicted by the boxes. Adjusted p*<0.05; **<0.01; ***<0.001.

### DETAILED DESCRIPTION

### Definitions

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art. Thus, while the following terms are believed to be well understood by one of ordinary skill in the art, the following definitions are set forth to facilitate explanation of the presently disclosed subject matter.

Throughout this specification, the word “comprise” or variations such as “comprises” or “comprising” will be understood to imply the inclusion of a stated component, or group of components, but not the exclusion of any other components, or group of components.

The term “a” or “an” refers to one or more of that entity, i.e. can refer to a plural referents. As such, the terms “a” or “an,” “one or more” and “at least one” are used interchangeably herein. In addition, reference to “an element” by the indefinite article “a” or “an” does not exclude the possibility that more than one of the elements is present, unless the context clearly requires that there is one and only one of the elements.

The term “including” is used to mean “including but not limited to.” “Including” and “including but not limited to” are used interchangeably.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the various novel and newly identified bacterial strains.

As used herein, “isolate,” “isolated,” “isolated microbe,” and like terms, are intended to mean that the one or more microorganisms has been separated from at least one of the materials with which it is associated in a particular environment (for example gastrointestinal fluid, gastrointestinal tissue, human digestive fluid, human digestive tissue, etc.). Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with a pharmaceutically acceptable carrier suitable for human administration.

In certain aspects of the disclosure, the isolated microbes exist as isolated and biologically pure cultures. As used herein the term “biologically pure” refers to a laboratory culture that is substantially free from other species of organism. Preferably, the bacterial species is in the form of a culture of a single species of organism. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state.

In certain aspects of the disclosure, the isolated microbes also encompass the use of variants or mutants of the bacterial species or strains described herein. As used herein, the terms “variant” and “mutant” include derived bacterial strains having at least 93% identity, at least 96% identity, at least 98%, or at least 99% identity to the genomic sequence of a referenced strain. Variants and mutants are obtainable by natural processes, mutagenesis campaigns, random culturing, and genetic engineering techniques, among others. The term “variant” is interchangeable herein with the term “mutant.”

As used herein, the term “mutations” includes natural or induced mutations comprising at least single base alterations including deletions, insertions, transversions, and other modifications known to those skilled in the art, including genetic modification introduced into a parent nucleotide or amino acid sequence.

As used herein, “individual isolates” refers to a composition or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

As used herein, “probiotic” refers to a substantially pure microbe (i.e., a single isolate) or a mixture of desired microbes, and may also include any additional components that can be administered to a subject (e.g., a human) for restoring or altering microbiota. Probiotics or microbial inoculant compositions of the disclosure may be administered with an agent to allow the microbes to survive the environment of the gastrointestinal tract, i.e., to resist low pH and to grow in the gastrointestinal environment. In some embodiments, the present compositions (e.g., microbial compositions) are probiotics.

As used herein, “prebiotic” refers to an agent that increases the number and/or activity of one or more desired microbes. Non-limiting examples of prebiotics that may be useful in the methods of the present disclosure include fructooligosaccharides (e.g., oligofructose, inulin, inulin-type fructans), galactooligosaccharides, amino acids, alcohols, and mixtures thereof. See Ramirez-Farias et al. (2008. Br. J Nutr. 4:1-10) and Pool-Zobel and Sauer (2007. J Nutr. 137:2580-2584 and supplemental).

As used herein, “live biotherapeutic product” or “LBP” refers to a biological product that: 1) contains live organisms, such as bacteria, and 2) is applicable to the prevention, treatment, or cure of a disease or condition of a subject in need thereof. In some embodiments, a LBP is a therapeutic composition which will undergo or has undergone clinical regulatory approval.

A “combination” of two or more bacteria includes the physical co-existence of the bacteria, either in the same material or product or in physically connected products, as well as the temporal co-administration or co-localization of the different bacteria.

The terms “percent identity” or “identity” in the context of two or more nucleic acids or polypeptides, refer to two or more sequences that are the same or have a specified percentage of nucleotides or amino acid residues that are the same. The percent identity can be measured using sequence comparison software or algorithms or by visual inspection.

In general, percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides refers to the minimum number of the 16S rRNA gene nucleotides that are necessary to align the second sequence, and does not include alignment (e.g., forced alignment) with non-16S rRNA gene sequences. The total number of aligned nucleotides may correspond to the entire the 16S rRNA gene sequence or may correspond to fragments of the full-length the 16S rRNA gene sequence.

Sequences can be aligned using the algorithm described by Altschul et al. (Nucleic Acids Res, 25:3389-3402, 1997) as incorporated into BLAST (basic local alignment search tool) programs, available at ncbi.nlm.nih.gov on the World Wide Web. BLAST searches or alignments can be performed to determine percent sequence identity between a 16S rRNA gene nucleic acid and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a 16S rRNA gene sequence and another sequence, the default parameters of the respective programs are used.

The phrases “substantially similar” and “substantially identical” in the context of at least two nucleic acids typically means that a polynucleotide comprises a sequence that has at least about 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% sequence identity, in comparison with a reference polynucleotide. In some embodiments, substantially identical nucleic acid molecules hybridize to each other under stringent conditions (e.g., within a range of medium to high stringency).

The primary structure of major rRNA subunit 16S comprises a particular combination of conserved, variable, and hypervariable regions that evolve at different rates and enable the resolution of both very ancient lineages such as domains, and more modern lineages such as genera. The secondary structure of the 16S subunit includes approximately 50 helices which result in base pairing of about 67% of the residues. These highly conserved secondary structural features are of great functional importance and can be used to ensure positional homology in multiple sequence alignments and phylogenetic analysis. Over the previous few decades, the 16S rRNA gene has become the most sequenced taxonomic marker and is the cornerstone for the current systematic classification of bacteria and archaea (Yarza et al. 2014. Nature Rev. Micro. 12: 635-45).

A sequence identity of 94.5% or lower for two 16S rRNA genes is strong evidence for distinct genera, 86.5% or lower is strong evidence for distinct families, 82% or lower is strong evidence for distinct orders, 78.5% is strong evidence for distinct classes, and 75% or lower is strong evidence for distinct phyla. The comparative analysis of 16S rRNA gene sequences enables the establishment of taxonomic thresholds that are useful not only for the classification of cultured microorganisms but also for the classification of the many environmental sequences. Yarza et al. 2014. Nature Rev. Micro. 12:635-45).

The term “relative abundance” as used herein, is the number or percentage of a microbe present in the gastrointestinal tract or other organ system, relative to the number or percentage of total microbes present in said tract or organ system. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including but not limited to array or microarray hybridization, quantitative PCR, and culturing and performance of colony forming unit assays (cfu, CFU) or plaque forming unit assays (pfu, PFU) performed on samples from the gastrointestinal tract or other organ system of interest.

The terms “patient,” “subject,” and “individual” may be used interchangeably and refer to either a human or a non-human animal. These terms include mammals such as humans, non-human primates, livestock animals (e.g., bovines, porcines, ovine, caprine, or poultry), companion animals (e.g., canines, felines, equine, or oryctolagus) and rodents (e.g., mice and rats). In certain embodiments, the terms refer to a human patient.

As used herein, “inhibiting and suppressing” and like terms should not be construed to require complete inhibition or suppression, although this may be desired in some embodiments. Thus, an “inhibited immune response” or the “inhibition of inflammatory cytokines” does not require absolute inhibition.

“Dysbiosis” refers to a state of the microbiota or microbiome of the gut or other body area, including mucosal or skin surfaces (or any other microbiota niche) in which the normal diversity and/or function of the ecological network is disrupted. Any disruption from the preferred (e.g., ideal.) state of the microbiota can be considered a dysbiosis, even if such dysbiosis does not result in a detectable decrease in health. This state of dysbiosis may be unhealthy (e.g., result in a diseased state), it may be unhealthy under only certain conditions, or it may prevent a subject from becoming healthier. Dysbiosis may be due to a decrease in diversity of the microbiota population composition, the overgrowth of one or more population of pathogens (e.g., a population of pathogenic bacteria) or pathobionts, the presence of and/or overgrowth of symbiotic organisms able to cause disease only when certain genetic and/or environmental conditions are present in a patient, or a shift to an ecological network that no longer provides a beneficial function to the host and therefore no longer promotes health. A state of dysbiosis may lead to a disease or disorder (e.g., a gastrointestinal disease, disorder or condition), or the state of dysbiosis may lead to a disease or disorder (e.g., a gastrointestinal disease, disorder or condition) only under certain conditions, or the state of dysbiosis may prevent a subject from responding to treatment or recovering from a disease or disorder (e.g., a gastrointestinal disease, disorder or condition).

The term “gut” as used herein refers to the entire gastrointestinal or digestive tract (also referred to as the alimentary canal) and it refers to the system of organs within multi-cellular animals which takes in food, digests it to extract energy and nutrients, and expels the remaining waste. As used herein the term “gastrointestinal tract” refers to the entire digestive canal, from the oral cavity to the rectum. The term “gastrointestinal tract” includes, but is not limited to, mouth and proceeds to the esophagus, stomach, small intestine, large intestine, rectum and, finally, the anus.

As used herein, the term “therapeutically effective amount” refers to an amount of a therapeutic agent (e.g., a microbe, live biotherapeutic product (LBP), and/or probiotic of the disclosure), which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of, or feels an effect). In some embodiments, a “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease.

As used herein, the term “treatment” (also “treat” or “treating”) refers to any administration of a therapeutic agent (e.g., a microbe, LBP, and/or probiotic of the disclosure), according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., chronic or recurring immune response and inflammation of the GI tract); in some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein, the term “medicament” encompasses medicaments for both human and animal usage in human and veterinary medicine. In addition, the term “medicament” as used herein means any substance, which provides a therapeutic and/or beneficial effect. The term “medicament” as used herein is not necessarily limited to substances that need Marketing Approval, but may include substances that can be used in cosmetics, nutraceuticals, food (including feeds and beverages for example), probiotic cultures, LBPs, nutritional supplements and natural remedies. In addition, the term “medicament” as used herein encompasses a product designed for incorporation in animal feed, for example livestock feed and/or pet food.

“Pharmaceutical” implies that a composition, microbe, reagent, method, and the like, are capable of a pharmaceutical effect, and also that the composition is capable of being administered to a subject safely. “Pharmaceutically acceptable” means approved by a regulatory agency of the Federal or a state government or listed in the U.S. Pharmacopoeia or other generally recognized pharmacopoeia for safe use in animals, and more particularly safe use in humans. “Pharmaceutically acceptable vehicle” or “pharmaceutically acceptable excipient” refers to a diluent, adjuvant, excipient or carrier with which a microbe as described herein is administered. The preparation of a pharmaceutical composition or additional active ingredient will be known to those of skill in the art in light of the present disclosure, as exemplified by Remington's Pharmaceutical Sciences, 18ₜₕ Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

The therapeutic pharmaceutical compositions taught herein may comprise one or more natural products, however, in certain embodiments, the therapeutic pharmaceutical compositions themselves do not occur in nature. Further, in certain embodiments, the therapeutic pharmaceutical compositions possess markedly different characteristics, as compared to any individual naturally occurring counterpart, or composition component, which may exist in nature. That is, in certain embodiments, the pharmaceutical compositions taught herein-which comprise a therapeutically effective amount of an isolated microbe-possess at least one structural and/or functional property that impart markedly different characteristics to the composition as a whole, as compared to any single individual component of the composition as it may exist naturally. The courts have determined that compositions comprising natural products, which possess markedly different characteristics as compared to any individual component as it may exist naturally, are statutory subject matter. Thus, the taught therapeutic pharmaceutical compositions as a whole possess markedly different characteristics. These characteristics are illustrated in the data and examples taught herein.

Details of the disclosure are set forth herein. Although methods and materials similar or equivalent to those described herein can be used in the practice or testing of the present disclosure, illustrative methods and materials are now described. Other features, objects, and advantages of the disclosure will be apparent from the description and from the claims.

### Dysbiosis in Inflammatory Bowel Diseases

The incidence of Inflammatory Bowel Disease (IBD) has dramatically increased in the developed countries in recent years. Crohn's disease (CD) and ulcerative colitis (UC) are the two main forms of IBD, which have distinct as well as overlapping pathologic and clinical characteristics. It is known that both genetic and environmental factors including impaired host immune response to the intestinal microbiota contribute to IBD. In past IBD studies, fecal samples are most frequently utilized to identify microbiota associated with IBD (Jacobs et al., 2016, Cell Mol Gastroenterol Hepatol, 2:750-766, Doherty et al., 2018, MBio, 9: e02120-17, Kennedy et al., 2018, Inflamm Bowel Dis, 24:583-592), however they do not always directly reflect the environment of the inflamed sites including histopathology of the intestinal mucosa, oxygen availability, and pH. Thus, in the present document, mucosal biopsies were collected from the inflamed intestinal tissue of patients with CD or UC, as well as from the non-IBD controls to characterize both the microbiota and host response from the same environment.

Many IBD studies focusing on mucosal microbiome have been published so far, however, their sample sizes were limited and only a handful of those have matched host response data. Kiely et al. (2018, Gut Microbes, 9:477-485) and Morgan et al. (2015, Genome Biol, 16:67) examined IBD microbiome in less than 100 samples and lacking in host response. Mottawea et al. (2016, Nature Communications, 7: 13419), Lepage et al. (2011, Gastroenterology, 141:227-236) and Hasler et al. (2017, Gut, 66:2087-2097) examined both microbiome and host, however, the cohort size was limited to up to 124. As described in Example 1, one of the largest adult IBD studies to date was conducted with 185 Irish subjects integrating both microbiota and host response data (RNAseq).

Although extensive effort has been taken to profile microbiota using the 16S rRNA gene from DNA samples (DNA-16S), this method only confirms bacterial abundance and not necessarily whether they are alive or metabolically active. Indeed, several studies have shown that active microbiota obtained by analyzing 16S rRNA gene expression levels in RNA samples (RNA-16S) are different from the traditional DNA-16S profiles (Perez-Cobas et al., 2013, Gut, 62:1591-1601; Bajaj et al., 2018, JCI Insight, 3: e98019; Ji et al., 2018, Front Microbiol, 9:710). Hasler et al. (2016) examined correlations between RNA-16S and host RNAseq, however, at the class level and DNA-16S was not compared. Accordingly, described herein are studies in which both RNA-16S and DNA-16S from mucosal samples of IBD patients were examined and compared to RNA-16S and DNA-16S from healthy counterparts to better understand associations between mucosal microbiota and intestinal disorders.

### Analyzing Bacterial Numbers and Metabolic Activity in IBD Versus Healthy Subjects

The study described in Example 1 below includes an analysis of one of the largest adult IBD mucosal microbiota cohorts published so far and examined bacterial associations with IBD using high-resolution denoised sequencing data from both DNA and RNA of 16S rRNA gene with strain-specific taxonomic classification where possible, in conjunction with host transcriptome datasets.

Numerous past studies have proposed bacterial associations with IBD, however, either the cohort sizes were considerably small and/or the results were based solely on DNA-16S. While DNA-16S sequencing is widely used, there are not many studies applying it to RNA and there appears to be no studies investigating IBD microbiota by a combination of both approaches.

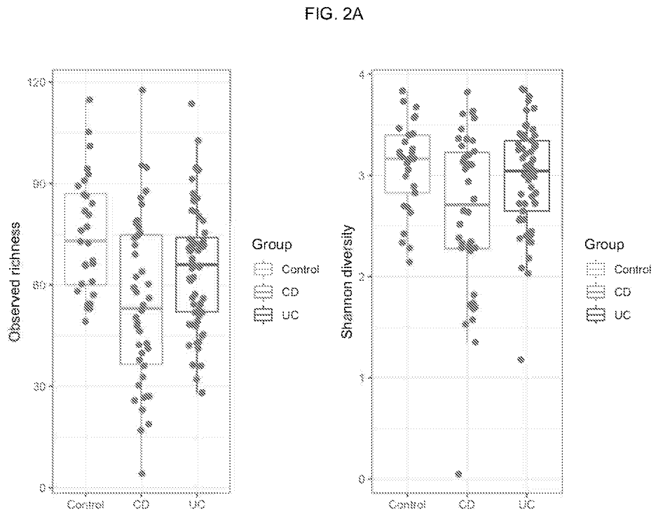

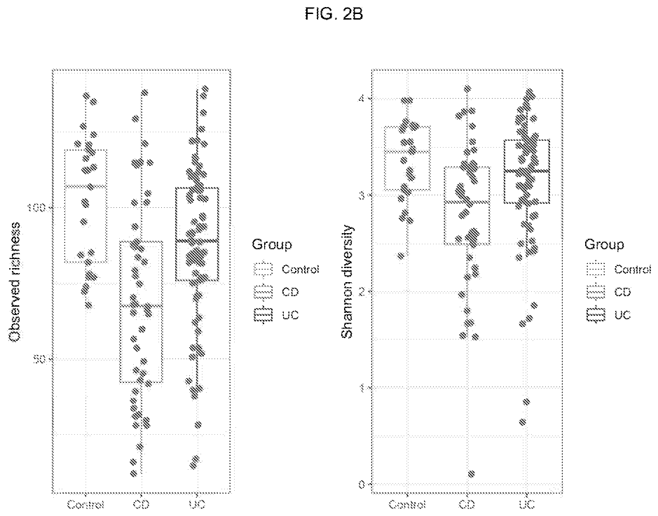

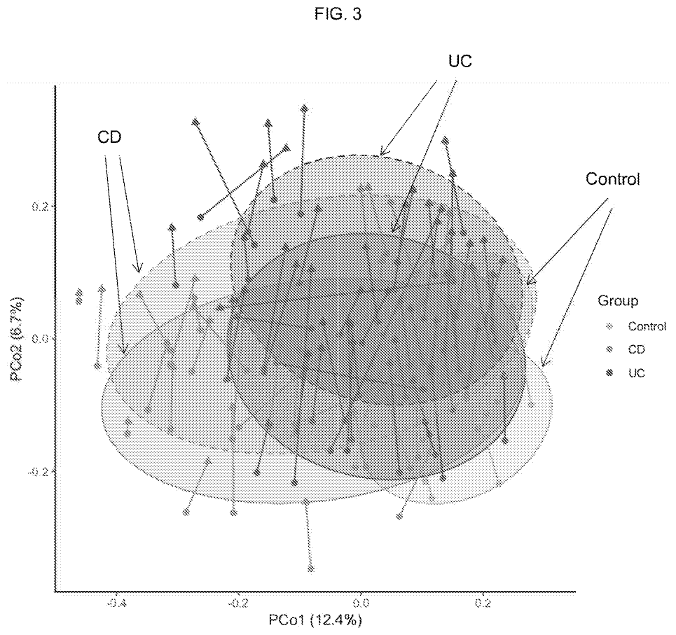

The results of the study, detailed in Example 1 below, demonstrate the commonly observed trend in alpha diversity metrics, wherein the microbiota of IBD patients possesses lower richness and diversity (FIGS. 2A and 2B). Theoretically, there should be fewer unique RNA-16S taxa than DNA-16S taxa as not all bacteria are metabolically active. However, in the present study, more unique RSVs in RNA-16S were observed compared to DNA-16S, which could be due to the deeper RNA-16S sequencing depth compared to that of DNA-16S (median 79,451 compared to 60,765). PERMANOVA analysis demonstrated significant differences between DNA-16S and RNA-16S, both by Bray-Curtis and Sorensen distances, which are depicted in PCoA plots (FIG. 3). Since Sorensen index is calculated by presence/absence, and not the abundance of RSVs, the results indicate a shift in PCoA could be due to additional RSVs identified in RNA-16S samples.

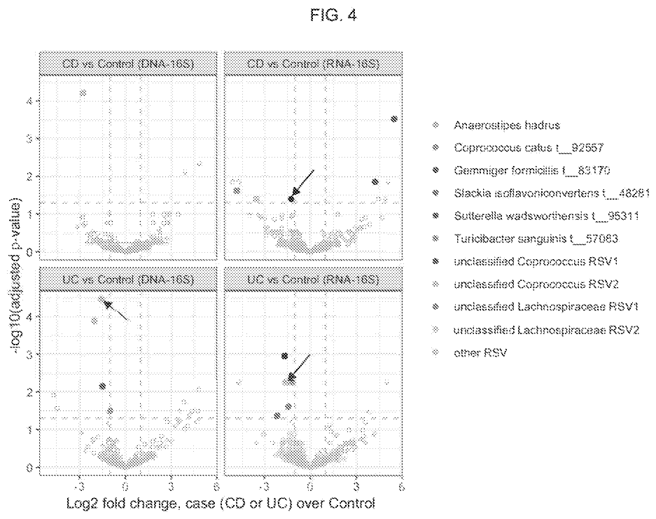

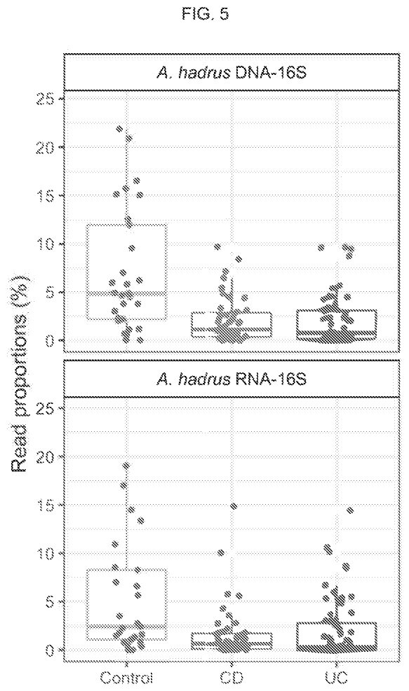

Traditionally, 16S rRNA sequencing results have been often analyzed using OTU clustering within the study (e.g., Mottawea et al., 2016, Nature Communications, 7: 13419; Jacobs et al., 2016, Cell Mol Gastroenterol Hepatol, 2: 750-766), which limits the ability to compare across different studies. To denoise sequencing errors and facilitate the comparability across studies, a DADA2 approach (Callahan et al., 2016, Natures Methods, 13:581-583) was employed, which produces counts of error corrected sequences in each sample. Differential abundance/expression tests revealed 10 dynamic RSVs across all four comparisons (FIG. 4). Age was identified as a possible confounding factor by ANOVA (p<0.001; Table 1). PERMANOVA indicated significant association with microbiota variance (p=0.021 in DNA-16S), however, none of the specific RSV demonstrated significant correlation. Thus, the identified RSVs associated with disease status are not affected by age. A Coprococcus species (FIG. 4) was depleted in both CD and UC for DNA-1 6S, and in UC for RNA-16S. On the other hand, another Coprococcus species (RSV1 in FIG. 4) was enriched in CD compared to controls in RNA-16S, which suggests different contribution of this genus to the microenvironment at species or strain level, further highlighting the importance of sub-genus classification. Decreased levels of Coprococcus were also reported in IBD samples by Chen et al. (2014, Medicine (Baltimore), 93: e51) and Gevers et al. (2014, Cell Host Microbe, 15:382-392). A. hadrus was depleted in UC for DNA-16S, and in both CD and UC for RNA-16S. Notably, both A. hadrus and Coprococcus are affiliated with the Lachnospiraceae family. Coprococcus GD/7 and G. formicilis X2-56 were depleted in UC in both DNA-16S and RNA-16S. Bajer and colleagues also reported low abundance of C. catus in the IBD patients (Bajer et al., 2017, World J Gastroenterol, 23:4548-4558), whereas high abundance of G. formicilis has been reported in the patients with recurrence of CD lesions (Mondot et al., 2015, Gut, 65:954-962), however UC patients were not examined in this study. Sutterella wadsworthensis 2_1_59BFAA was enriched in CD in RNA-16S. Sutterella species have been investigated for association to IBD in many studies (Hiippala et al., 2016, Front Microbial, 7:1706; Mangin et al., 2004, Mukhopadhya et al., 2011), however, no clear differences have been shown between the IBD patients and controls. In UC versus control comparisons, the four RSVs identified differentially abundant in DNA-16S dataset were a subset of those in RNA-16S dataset. On the other hand, in CD versus control comparisons, none of the enriched or depleted RSVs overlapped between DNA and RNA datasets.

As a follow-up to these findings, A. hadrus was further explored as it was depleted in three out of four comparisons: UC versus controls in DNA-16S, and both UC and CD versus controls in RNA-16S. This Gram-positive bacterium, one of the most dominant bacterium in the human colon, is known to have butyrate-producing capabilities (Allen-Vercoe et al., 1976, Anaerobe, 18:523-529) that are reported to be beneficial by many studies (Canani et al. 2011, World J Gastroenterol, 17: 1519-1528; Geirnaert et al., 2017, Sci Rep, 7: 11450). Although the A. hadrus RSV was identified by comparing UC and CD with controls, a majority of the differentially expressed host genes associated with the A. hadrus RSV were not significantly up/down-regulated in UC or CD. The same trend was observed at pathway level, where termination of O-glycan biosynthesis pathway was most significantly enriched when contrasting A. hadrus RSV to host RNAseq, but not when contrasted CD or UC to controls (data not shown). This could be due to the heterogeneous nature of IBD; the interaction between specific bacterium and host gene expression may address association of gut microbiota to the disease more directly.

O-glycans, in particular mucin, are important for the development of physiological environment for intestinal microflora. MUC6, MUC12 and MUC13 encode for mucin glycoproteins that protect the gut lumen by forming an insoluble mucous barrier. O-glycan biosynthesis can be terminated via sialic acid (Varki et al., 2009, Essentials of Glycobiology, 2^{nd} edition, Cold Spring Harbor Laboratory Press) that attaches to the O-glycans and modulates voltage-gated potassium channel gating (Schwetz et al., 2011, J Biol Chem, 286:4123-4132). Terminal modification of glycans are known to affect bacterial adhesion (Baos et al., 2012, Biophys J, 102:176-184) and could serve as substrates that provide nutritional advantage to specific microbes (Pacheco et al., 2012, Nature, 491:113-117). A. hadrus could be a glycan degrader and/or a butyrate producer in a cross-feeding network with glycan degradation to maintain mucosal homeostasis as has been reported for Akkermansia muciniphila and other butyrate producers including Anaerostipes caccae (Ouwerkerk et al., 2013, Best Pract Res Clin Gastroenterol, 27:25-38; Belzer et al., 2017, MBio, 8: e00770-1 7). In the present study, A. muciniphila was detected in both DNA-16S and RNA-16S datasets, albeit below the significance threshold.

Bioinformatics tool benchmarking was not an intention of the study described herein, but apart from metagenomeSeq, also explored was a differential test with DESeq2 (Love et al., 2014, Genome Biology, 15:550). DESeq2 is primarily designed for the differential gene expression analysis of RNAseq data, which is less sparse than marker-gene abundances (Paulson et al., 2013, metagenomeSeq: Statistical analysis for sparse high-throughput sequencing. Bioconductor package: 1.11.10 ed. 2013). The sparsity of the datasets after prevalence filtering was 79.5% for DNA-16S and 81.0% for RNA-16S. Even though the sparsities were comparable to the RNAseq data, DESeq2 resulted in fewer significant findings compared to metagenomeSeq. In particular, DESeq2 was insensitive to the differential expression of the A. hadrus RSV in either of the UC or CD comparison against controls in RNA-16S (adjusted p=0.13 for CD or 0.37 for UC compared to controls). As relative abundance boxplots confirmed different expression levels of A. hadrus in case (CD or UC) and controls (FIG. 5), in this particular dataset, it was decided to use metagenomeSeq over DESeq2 to identify significantly changed RSVs.

In summary, by employing an RNA-16S approach, it was determined that an A. hadrus RSV was depleted in both CD and UC relative to healthy controls. Moreover, direct comparison between A. hadrus and host gene expression identified significant enrichment of the termination of O-glycan biosynthesis pathway, which could lend an advantage to this microbe to survive and prosper in this environment, producing anti-inflammatory short chain fatty acids (SCFAs). Accordingly, provided herein are compositions and methods for treating an intestinal disease using a strain of A. hadrus or a variant thereof.

Anaerostipes hadrus (A. hadrus)

Anaerostipes hadrus is a gram positive bacterium which has been isolated from human feces (Allen-Vercoe et all, 2012, Anaerobe, 18:523-9). A complete genomic sequences for A. hadrusis available at the GenBank database (world wide web at ncbi.nlm.nih.gov/nuccore) as, e.g., Accession No. NZ_CP012098 (strain BPBS). Also available in the GenBank database are A. hadrus 16S rRNA sequences, published as GenBank Accession Nos. JF412658 (strain 5/1/63FAA; SEQ ID NO:1), NR_117139 (strain DSM 3319; SEQ ID NO:2), NR_117138 (strain DSM 3319; SEQ ID NO:3), NR_104799 (strain DSM 3319; SEQ ID NO:4), MG680450 (strain ASD1240; SEQ ID NO:5), AY305320 (strain SSC/2; SEQ ID NO:6), and AY305319 (SEQ ID NO:7) Accordingly, the present disclosure is directed to compositions and methods related to A. hadrus or variants thereof, wherein the A. hadrus strain has a genomic sequence which is at least about 95%, 96%, 97%, 98%, 99%, 99.1%, 99.2%, 99.3%, 99.4%, 99.5%, 99.6%, 99.7%, 99.8%, or 99.9% identical to the genomic sequence published as NZ_CP012098. Generally, a bacterial strain genomic sequence will contain multiple copies of 16S rRNA sequences. The 16S rRNA sequences are often used for making distinctions between species and strains, in that if one or more of the aforementioned sequences shares less than a specified % sequence identity from a reference sequence, then the two organisms from which the sequences were obtained are said to be of different species or strains. Accordingly, contemplated herein is a bacterium which comprises one or more 16S rRNA genes, each of which is at least about 95%, 96%, 97%, 98%, 99%, 99.1%, 99.2%, 99.3%, 99.4%, 99.5%, 99.6%, 99.7%, 99.8%, 99.9%, or 100% identical to SEQ ID NO:1, SEQ ID NO:2, SEQ ID NO:3, SEQ ID NO:4, SEQ ID NO:5, SEQ ID NO:6, or SEQ ID NO:7 and its use for treating a subject diagnosed with or at risk of developing an intestinal disease or disorder including but not limited to UC and CD. Also contemplated are compositions comprising a synthetic bacterium that has an engineered genome which has the same therapeutic efficacy as A. hadrus as described herein. In some embodiments, such a microbe has a genome which is at least about 95%, 96%, 97%, 98%, 99%, 99.1%, 99.2%, 99.3%, 99.4%, 99.5%, 99.6%, 99.7%, 99.8%, 99.9%, or 100% identical to the genome of GenBank Acc. No. NZ_CP012098. In some embodiments, the compositions as provided herein comprise a population of the bacterium (i.e., a population of the strain of A. hadrus or a variant thereof), wherein each member of the population has a substantially identical 16S rRNA gene sequence.

Therapeutic Uses for A. hadrus

Provided herein are methods for treating a subject in need thereof comprising administering to the subject a composition comprising a strain of A. hadrus or a variant thereof as described in the present disclosure. In some embodiments, the compositions as provided herein comprise a population of the bacterium (i.e., a population of the strain of A. hadrus or a variant thereof), wherein each member of the population has a substantially identical 16S rRNA gene sequence. The subject can be one who has been diagnosed with inflammatory bowel disease, ulcerative colitis, pediatric UC, Crohn's disease, pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome, or chemotherapy associated steatohepatitis (CASH).

### Inflammatory Bowel Disease

Inflammatory bowel disease (IBD) classically includes ulcerative colitis (UC) and Crohn's disease (CD). The pathogenesis of inflammatory bowel disease is not known. A genetic predisposition has been suggested, and a host of environmental factors, including bacterial, viral and, perhaps, dietary antigens, can trigger an ongoing enteric inflammatory cascade. IBD can cause severe diarrhea, pain, fatigue, and weight loss. IBD can be debilitating and sometimes leads to life-threatening complications. Accordingly, in some embodiments, the method of treatment as described herein is effective to reduce, prevent or eliminate any one or more of the symptoms described above wherein the method comprises administering to a patient in need thereof a therapeutically effective amount of a composition comprising a strain of A. hadrus. In some embodiments, the method of treatment results in remission.

### Ulcerative Colitis

Ulcerative colitis is an inflammatory bowel disease that causes long-lasting inflammation and sores (ulcers), in the innermost lining of the large intestine (colon) and rectum. Ulcerative colitis typically presents with shallow, continuous inflammation extending from the rectum proximally to include, in many patients, the entire colon. Fistulas, fissures, abscesses and small-bowel involvement are absent. Patients with limited disease (e.g., proctitis) typically have mild but frequently recurrent symptoms, while patients with pancolitis more commonly have severe symptoms, often requiring hospitalization. Botoman et al., “Management of Inflammatory Bowel Disease,” Am. Fam. Physician, Vol. 57(1):57-68 (Jan. 1, 1998) (internal citations omitted). Thus, ulcerative colitis is an IBD that causes long-lasting inflammation and sores (ulcers) in the innermost lining of your large intestine (colon) and rectum.

### Crohn's Disease

Unlike ulcerative colitis, Crohn's disease can involve the entire intestinal tract, from the mouth to the anus, with discontinuous focal ulceration, fistula formation and perianal involvement. The terminal ileum is most commonly affected, usually with variable degrees of colonic involvement. Subsets of patients have perianal disease with fissures and fistula formation. Only 2 to 3 percent of patients with Crohn's disease have clinically significant involvement of the upper gastrointestinal tract. Botoman et al., “Management of Inflammatory Bowel Disease,” Am. Fam. Physician, Vol. 57(1):57-68 (Jan. 1, 1998) (internal citations omitted). Thus, Crohn's disease is an IBD that causes inflammation of the lining of your digestive tract. In Crohn's disease, inflammation often spreads deep into affected tissues. The inflammation can involve different areas of the digestive tract, i.e., the large intestine, small intestine, or both. Collagenous colitis and lymphocytic colitis also are considered inflammatory bowel diseases, but are usually regarded separately from classic inflammatory bowel disease.

### Clinical Parameters of Inflammatory Bowel Disease

As previously discussed, inflammatory bowel disease encompasses ulcerative colitis and Crohn's disease. There are numerous scores and clinical markers known to one of skill in the art that can be utilized to access the efficacy of the administered A. hadrus compositions described herein in treating these conditions.

There are two general approaches to evaluating patients with IBD. The first involves the visual examination of the mucosa and relies on the observation of signs of damage to the mucosa, in view of the fact that IBD is manifested by the appearance of inflammation and ulcers in the GI tract. Any procedure that allows an assessment of the mucosa can be used. Examples include barium enemas, x-rays, and endoscopy. An endoscopy may be of the esophagus, stomach and duodenum (esophagogastroduodenoscopy), small intestine (enteroscopy), or large intestine/colon (colonoscopy, sigmoidoscopy). These techniques are used to identify areas of inflammation, ulcers and abnormal growths such as polyps.

Scoring systems based on this visual examination of the GI tract exist to determine the status and severity of IBD, and these scoring systems are intended to ensure that uniform assessment of different patients occurs, despite the fact that patients may be assessed by different medical professionals, in diagnosis and monitoring of these diseases as well as in clinical research evaluations. Examples of evaluations based on visual examination of UC are discussed and compared in Daperno M et al (J Crohns Colitis. 2011 5:484-98).

Clinical scoring systems also exist, with the same purpose. The findings on endoscopy or other examination of the mucosa can be incorporated into these clinical scoring systems, but these scoring systems also incorporate data based on symptoms such as stool frequency, rectal bleeding and physician's global assessment. IBD has a variety of symptoms that affect quality of life, so certain of these scoring systems also take into account a quantitative assessment of the effect on quality of life as well as the quantification of symptoms.

One example of a scoring system for UC is the Mayo scoring system (Schroeder et al., N Eng J Med, 1987, 317:1625-1629), but others exist that have less commonly been used and include the Ulcerative Colitis Endoscopic Index of Severity (UCEIS) score (Travis et al, 2012, Gut, 61:535-542), Baron Score (Baron et al., 1964, BMJ, 1:89), Ulcerative Colitis Colonoscopic Index of Severity (UCCIS) (Thia et al., 2011, Inflamm Bowel Dis, 17:1757-1764), Rachmilewitz Endoscopic Index (Rachmilewitz, 1989, BMJ, 298:82-86), Sutherland Index (also known as the UC Disease Activity Index (UCDAI) scoring system; Sutherland et al., 1987, Gastroenterology, 92:1994-1998), Matts Score (Matts, 1961, QJM, 30:393-407), and Blackstone Index (Blackstone, 1984, Inflammatory bowel disease. In: Blackstone Mo. (ed.) Endoscopic interpretation: normal and pathologic appearances of the gastrointestinal tract, 1984, pp. 464-494). For a review, see Paine, 2014, Gastroenterol Rep 2:161-168. Accordingly, also contemplated herein is a method for treating a subject diagnosed with and suffering from UC, wherein the treatment comprises administering A. hadrus as described herein and wherein the treatment results in a decrease in the UC pathology as determined by measurement of the UCEIS score, the Baron score, the UCCIS score, the Rachmilewitz Endoscopic Index, the Sutherland Index, and/or the Blackstone Index.

An example of a scoring system for CD is the Crohn's Disease Activity Index (CDAI) (Sands B et al 2004, N Engl J Med 350 (9): 876-85); most major studies use the CDAI in order to define response or remission of disease. Calculation of the CDAI score includes scoring of the number of liquid stools over 7 days, instances and severity of abdominal pain over 7 days, general well-being over 7 days, extraintestinal complications (e.g., arthritis/arthralgia, iritis/uveitis, erythema nodosum, pyoderma gangrenosum, aphtous stomatitis, anal fissure/fistula/abscess, and/or fever>37.8° C.), use of antidiarrheal drugs over 7 days, present of abdominal mass, hematocrit, and body weight as a ratio of ideal/observed or percentage deviation from standard weight. Based on the CDAI score, the CD is classified as either asymptomatic remission (0 to 149 points), mildly to moderately active CD (150 to 220 points), moderately to severely active CD (221 to 450 points), or severely active fulminant disease (451 to 1000 points). In some embodiments, the method of treatment comprising administering to a patient diagnosed with CD a composition comprising a therapeutically effective amount of A. hadrus results in a decrease in a diagnostic score of CD. For example, the score may change the diagnosis from severely active to mildly or moderately active or to asymptomatic remission.

The Harvey-Bradshaw index is a simpler version of the CDAI which consists of only clinical parameters (Harvey et al., 1980, Lancet 1(8178):1134-1135). The impact on quality of life is also addressed by the Inflammatory Bowel Disease Questionnaire (IBDQ) (Irvine et al., 1994, Gastroenterology 106: 287-296). Alternative methods further include CDEIS and SES CD (see, e.g., Levesque, et al. (2015) Gastroentrol. 148:37 57).

In some embodiments, a method of treating an IBD, e.g., UC, is provided wherein the treatment is effective in reducing the Mayo Score. The Mayo Score is a combined endoscopic and clinical scale used to assess the severity of UC and has a scale of 1-12 The Mayo Score is a composite of subscores for stool frequency, rectal bleeding, findings of flexible proctosigmoidoscopy or colonoscopy, and physician's global assessment (Paine, 2014, Gastroenterol Rep 2:161-168). With respect to rectal bleeding, blood streaks seen in the stool less than half the time is assigned 1 point, blood in most stools is assigned 2 points and pure blood passed is assigned 3 points. Regarding stool frequency, a normal number of daily stools is assigned 0 points, 1 or 2 more stools than normal is assigned 1 point, 3 or 4 more stools than normal is assigned 2 points, and 5 or more stools than usual is assigned 3 points. With respect to the endoscopy component, a score of 0 indicates normal mucosa or inactive UC, a score of 1 is given for mild disease with evidence of mild friability, reduced vascular pattern, and mucosal erythema, a score of 2 is given for moderate disease with friability, erosions, complete loss of vascular pattern, and significant erythema, and a score of 3 is given for ulceration and spontaneous bleeding (Schroeder et al., 1987, N Engl J Med, 317:1625-1629). Global assessment by a physician assigns 0 points for a finding of normal, 1 point for mild colitis, 2 points for moderate colitis and 3 points for severe colitis. Accordingly, in some embodiments, a patient treated with A. hadrus is successfully treated when the patient experiences a reduction in the Mayo Score by at least 1, 2 or 3 points in at least one of: rectal bleeding, blood streaks seen in the stool, endoscopy subscore and physician's global assessment. In some embodiments, the method of treatment comprising administering to a patient diagnosed with UC a therapeutically effective amount of A. hadrus results in a decrease in a diagnostic score of UC. For example, the score may change a diagnostic score, e.g., Mayo Score, by at least 1, 2, 3, 4, 5, 6, 7, 8, 9, 10 or 11 points.

### Pouchitis

Additionally or alternatively, the compositions comprising a strain of A. hadrus and methods of administration as described herein can be used to treat pouchitis. Pouchitis is an inflammation of the lining of a pouch that is surgically created in the treatment of UC. Specifically, subjects having serious UC may have their diseased colon removed and the bowel reconnected by a procedure called ileoanal anastomosis (IPAA) or J-pouch surgery. Pouchitis cases can recur in many patients, manifesting either as acute relapsing pouchitis or chronic, unremitting pouchitis. Accordingly, provided herein are methods for treating pouchitis, acute pouchitis or recurrent pouchitis.

Pouchitis activity can be classified as in remission (no active pouchitis), mild to moderately active (increased stool frequency, urgency, and/or infrequent incontinence), or severely active (frequent incontinence and/or the patient is hospitalized for dehydration). The duration of pouchitis can be defined as acute (less than or equal to four weeks) or chronic (four weeks or more) and the pattern classified as infrequent (1-2 acute episodes), relapsing (three or fewer episodes) or continuous. The response to medical treatment can be labeled as treatment responsive or treatment refractory, with the medication for either case being specified. Accordingly, in some embodiments, a method for treating a subject diagnosed with pouchitis is provided wherein treatment with a composition comprising A. hadrus results in a decrease in the severity of the pouchitis and/or results in remission.

### Mucositis and Mucosal Barriers

The mucosa of the GI tract is a complex microenvironment involving an epithelial barrier, immune cells, and microbes. A delicate balance is maintained in the healthy colon. Luminal microbes are physically separated from the host immune system by a barrier consisting of epithelium and mucus. The pathogenesis of IBD, although not fully elucidated, may involve an inappropriate host response to an altered commensal flora with a dysfunctional mucous barrier. See, Boltin et al., “Mucin Function in Inflammatory Bowel Disease an Update,” J. Clin. Gastroenterol., Vol. 47(2):106-111 (Feb. 2013).

Mucositis occurs when cancer treatments (particularly chemotherapy and radiation) break down the rapidly divided epithelial cells lining the intestinal tract (which goes from the mouth to the anus), leaving the mucosal tissue open to ulceration and infection. Mucosal tissue, also known as mucosa or the mucous membrane, lines all body passages that communicate with the air, such as the respiratory and alimentary tracts, and have cells and associated glands that secrete mucus. The part of this lining that covers the mouth, called the oral mucosa, is one of the most sensitive parts of the body and is particularly vulnerable to chemotherapy and radiation. The oral cavity is the most common location for mucositis. While the oral mucosa is the most frequent site of mucosal toxicity and resultant mucositis, it is understood that mucositis can also occur along the entire alimentary tract including the esophagus, stomach, small intestine (duodenum, jejunum, ileum), large intestine (colon), and rectum. In some embodiments, a composition comprising A. hadrus is therapeutically effective to treat mucositis of the mouth, esophagus, stomach, small intestine (duodenum, jejunum, ileum), large intestine (colon), and/or rectum.

Oral mucositis can lead to several problems, including pain, nutritional problems as a result of inability to eat, and increased risk of infection due to open sores in the mucosa. It has a significant effect on the patient's quality of life and can be dose-limiting (i.e., requiring a reduction in subsequent chemotherapy doses). The World Health Organization has an oral toxicity scale for diagnosis of oral mucositis: Grade 1: soreness±erythema, Grade 2: erythema, ulcers; patient can swallow solid food; Grade 3: ulcers with extensive erythema; patient cannot swallow solid food; Grade 4: mucositis to the extent that alimentation is not possible. Grade 3 and Grade 4 oral mucositis is considered severe mucositis. Accordingly, provided herein is a method for treating a subject diagnosed with oral mucositis, wherein administration of a composition comprising A. hadrus reduces the grade of oral toxicity by at least 1 point of the grade scale of 1 to 4.

In any of the above embodiments, a subject administered a composition as provided herein can experience a decrease in weight loss associated with inflammatory bowel disease, ulcerative colitis, pediatric UC, Crohn's disease, pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome, or chemotherapy associated steatohepatitis (CASH). In some embodiments, the weight loss of the subject after administration of the composition is less than that of a comparable patient who did not receive administration of the composition. For example, the weight loss of the subject after administration of the composition is about 5 lbs, 10 lbs, 15 lbs, 20 lbs, 25 lbs or 30 lbs less than that of a comparable patient who did not receive administration of the composition. In some embodiments, the weight loss of the subject is measured at 3 months, 4 months, 5 months, 6 months, 7 months, 8 months, 9 months, 10 months, 11 months, or 12 months after the first administration of the composition.

Compositions Comprising A. hadrus

The microbe compositions of the present disclosure can be administered to a subject in need thereof to enhance general health and well-being and/or to treat or prevent a disease or disorder such as a metabolic disorder as described herein. In some embodiments, the microbe composition is a live bacterial product (LBP) while in other embodiments, the microbe composition is a probiotic. In some embodiments, the bacterium (A. hadrus) is isolated and has been cultured outside of an animal to increase the number or concentration of the microbes, thereby enhancing the therapeutic efficacy of a composition comprising the microbe population. In some embodiments, the compositions as provided herein comprise a population of the bacterium (i.e., a population of the strain of A. hadrus or a variant thereof), wherein each member of the population has a substantially identical 16S rRNA gene sequence.

In some embodiments, the microbe composition is in the form of a live bacterial population. The live population may be, e.g., frozen, cryoprotected or lyophilized. In other embodiments, the microbe composition comprises a non-viable bacterial preparation, or the cellular components thereof. In some embodiments, where the microbe composition is in the form of a non-viable bacterial preparation, it is selected from, for example, heat-killed bacteria, irradiated bacteria and lysed bacteria.

In some embodiments, the bacterial species is in biologically pure form, substantially free from other species of organism. In some embodiments, the bacterial species is in the form of a culture of a single species of organism.

Compositions comprising A. hadrus in accordance with the present disclosure can be any of a number of accepted probiotic or LBP delivery systems suitable for administration to a subject. Importantly, a composition for delivery of a live population of A. hadrus must be formulated to maintain viability of the microbes. In some embodiments, the composition comprises elements which protect the bacteria from the acidic environment of the stomach. In some embodiments, the composition includes an enteric coating.

In some embodiments, the composition is a food-based product. A food-based product can be, for example, a yogurt, cheese, milk, meat, cream, or chocolate. Such food-based products can be considered edible, which means that it is approved for human or animal consumption.

One aspect of the disclosure relates to a food product comprising the bacterial species defined above. The term “food product” is intended to cover all consumable products that can be solid, jellied or liquid. Suitable food products may include, for example, functional food products, food compositions, pet food, livestock feed, health foods, feedstuffs, and the like. In some embodiments, the food product is a prescribed health food.

As used herein, the term “functional food product” means food that is capable of providing not only a nutritional effect, but is also capable of delivering a further beneficial effect to the consumer. Accordingly, functional foods are ordinary foods that have components or ingredients (such as those described herein) incorporated into them that impart to the food a specific functional—e.g. medical or physiological benefit—other than a purely nutritional effect.

Examples of specific food products that are applicable to the present disclosure include milk-based products, ready to eat desserts, powders for re-constitution with, e.g., milk or water, chocolate milk drinks, malt drinks, ready-to-eat dishes, instant dishes or drinks for humans or food compositions representing a complete or a partial diet intended for humans, pets, or livestock.

In one embodiment, the composition according to the present disclosure is a food product intended for humans, pets or livestock. The composition may be intended for animals selected from the group consisting of non-human primates, dogs, cats, pigs, cattle, horses, goats, sheep, or poultry. In another embodiment, the composition is a food product intended for adult species, in particular human adults.

Another aspect of the disclosure relates to food products, dietary supplements, nutraceuticals, nutritional formulae, drinks and medicaments containing the bacterial species as defined above, and use thereof.

In the present disclosure, “milk-based product” means any liquid or semi-solid milk or whey based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavored milks, ice cream; milk-containing food such as sweets.

The microbe composition can be a tablet, a chewable tablet, a capsule, a stick pack, a powder, or effervescent powder. The composition can comprise coated beads which contain the bacteria. A powder may be suspended or dissolved in a drinkable liquid such as water for administration.

In some embodiments, the microbe composition comprises a microbe which is isolated. The isolated microbe may be included in a composition with one or more additional substance(s). For example, the isolated microbe may be included in a pharmaceutical composition with one or more pharmaceutically acceptable excipient(s). Prior to formulating a pharmaceutical composition containing the isolated microbe, the isolated microbe may be culture in vitro to increase the population of the microbe.

In some embodiments, the microbe composition may be used to promote or improve human health. In some aspects, the microbe composition may be used to improve gut health. In some aspects, the microbe composition may be used to regulate appetite. In some aspects, the microbe composition may be used to regulate blood glucose levels. In some aspects, the microbe composition may be used to regulate insulin sensitivity

In some embodiments, the disclosed microbe composition is used for regulating appetite in a subject.

The microbes described herein may also be used in prophylactic applications. In prophylactic applications, bacterial species or compositions according to the disclosure are administered to a patient susceptible to, or otherwise at risk of, a particular disease in an amount that is sufficient to at least partially reduce the risk of developing a disease. The precise amounts depend on a number of patient specific factors such as the patient's state of health and weight.

Also provided herein are methods for maintaining or improving overall fitness or health of a subject is provided herein that comprise administering to a subject a therapeutic composition comprising a bacterium (e.g., A. Hadrus) which has a 16S rRNA gene that is at least 75%, 80%, or 85% identical to SEQ ID NO:1, SEQ ID NO:2, SEQ ID NO:3, SEQ ID NO:4, SEQ ID NO:5, SEQ ID NO:6 or SEQ ID NO:7. The compositions as provided herein typically comprises a population of the bacterium, wherein each member of the population has a substantially identical 16S rRNA gene sequence. In some embodiments, the composition further comprises a pharmaceutically acceptable excipient. In some embodiments, the overall fitness or health of the patient after administration of the composition is greater than that of a comparable patient who did not receive administration of the composition. Overall fitness or health can be assessed using methods and techniques recognized in the art. For example, benefits of administration of compositions as provided herein and linked to overall fitness and health can include reduction of pathogen load, improved microbial fermentation patterns, improved nutrient absorption, improved immune function, improved intestinal hormonal signaling and metabolic regulation, aided digestion, increasing training endurance or performance endurance, reducing lactate levels in a human during or after physical activity generating increased lactate levels, reducing inflammation within a human resulting from physical activity, increasing energy metabolism within a human during physical activity, improving athletic training, performance or recovery by a human during physical activity, recovering from physical activity resulting in inflammation and increased lactate levels, or promoting weight loss.

In some aspects, the disclosure provides for various immediate and controlled release formulations comprising the microbes described herein and combinations thereof. Controlled release formulations sometimes involve a controlled release coating disposed over the bacteria. In particular embodiments, the controlled release coatings may be enteric coatings, semi-enteric coatings, delayed release coatings, or pulsed release coatings may be desired. In particular, a coating will be suitable if it provides an appropriate lag in active release (i.e. release of the therapeutic microbes and combinations thereof). It can be appreciated that in some embodiments one does not desire the therapeutic microbes and combinations thereof to be released into the acidic environment of the stomach, which could potentially degrade and/or destroy the taught microbes, before it reaches a desired target in the intestines.

In some embodiments, the microbe compositions of this disclosure encompass A. hadrus and any variants thereof as described above.

In some embodiments, the pharmaceutical microbe composition of the present disclosure further comprises a prebiotic in an amount of from about 1 to about 30% by weight, respect to the total weight composition. For example, the prebiotic can be from 5 to 20% by weight of the total weight of the composition. Exemplary carbohydrates are selected from: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. For example, in one embodiment, prebiotics can be short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

In one embodiment, the composition further comprises at least one other kind of other food grade bacterium, wherein the food grade bacterium is preferably selected from the group consisting of lactic acid bacteria, bifidobacteria, propionibacteria or mixtures thereof.

In some embodiments, microbe compositions comprise 10⁶-10¹² CFU, (colony forming units), 10⁸-10¹² CFU, 10¹⁰-10¹² CFU, 10⁸-10¹⁰ CFU, or 10⁸-10¹¹ CFU A. hadrus. In other embodiments, microbial combinations comprise about 10⁶, about 10⁷, about 10⁸ about 10⁹, about 10¹⁰, about 10¹¹, or about 10¹² CFU A. hadrus.

### Administration

A composition comprising A. hadrus according to the present disclosure can be formulated for delivery to a desired site of action within an individual to whom it is administered. For example, the composition may be formulated for administration to the gastrointestinal lumen, or for delayed release in the intestine, terminal ileum, or colon.

When employed as a pharmaceutical, i.e., for treatment or prophylaxis of a disease or condition, the compositions described herein are typically administered in the form of a pharmaceutical composition. Such compositions can be prepared in a manner well known in the pharmaceutical art and include at least one active agent, i.e., a viable bacterium as described herein. Generally, the compositions are administered in a pharmaceutically effective amount, i.e., a therapeutically or prophylactically effective amount. The amount of the active agent, i.e., a microbe as described herein, administered will typically be determined by a physician, in the light of the relevant circumstances, including the condition to be treated, the chosen route of administration, the activity of the microbe or microbes administered, the age, weight, and response of the individual patient, the severity of the patient's symptoms, and the like.

The compositions for oral administration can take the form of bulk liquid solutions or suspensions, or bulk powders. More commonly, however, the compositions are presented in unit dosage forms to facilitate accurate dosing. Typical unit dosage forms include prefilled, premeasured ampules or syringes of the liquid compositions or pills, tablets, capsules or the like in the case of solid compositions.

The above-described components for orally administrable, or injectable administrable compositions are merely representative. Other materials, as well as processing techniques and the like are set forth in Part 8 of Remington's The Science and Practice of Pharmacy, 21^{st} edition, 2005, Publisher: Lippincott Williams & Wilkins, which is incorporated herein by reference.

For oral administration, particular use is made of compressed tablets, pills, tablets, gellules, drops, and capsules.

In another embodiment, the compositions of the disclosure are administered in combination with one or more other active agents. In such cases, the compositions of the disclosure may be administered consecutively, simultaneously or sequentially with the one or more other active agents.

### Dosage and Administration Schedule

The dosages disclosed herein are exemplary of the average case. There can of course be individual instances where higher or lower dosage ranges are merited, and such are within the scope of this invention.

In some embodiments, the effective daily dose in a subject can be from about 1×10⁶ to about 1×10¹² colony forming units (CFUs), 1×10⁷ to 1×10¹² CFUs, 1×10⁸ to 1×10¹² CFUs, 1×10⁸ to 1×10¹¹ CFUs, 1×10⁸ to 1×10¹⁰ CFUs, 1×10⁸ to 1×10⁹ CFUs, 1×10⁹ to 1×10¹² CFUs, 1×10¹⁰ to 1×10¹² CFUs, or 1×10¹⁰ to 1×10¹¹ CFUs. The subject may be a human or non-human primate. Alternatively, the subject may be another mammal such as a rat, mouse, rabbit, etc.

In some embodiments, the daily dose can be administered to the subject daily for about 1 to 2 weeks, 1 to 4 weeks, 1 to 2 months, 1 to 6 months, or 1 to 12 months.

Alternatively, the dose, which can range from about 1×10⁶ to about 1×10¹² CFUs, 1×10⁷ to 1×10¹² CFUs, 1×10⁸ to 1×10¹² CFUs, 1×10⁸ to 1×10¹¹ CFUs, 1×10⁸ to 1×10¹⁰ CFUs, 1×10⁸ to 1×10⁹ CFUs, 1×10⁹ to 1×10¹² CFUs, 1×10¹⁰ to 1×10¹² CFUs, or 1×10¹⁰ to 1×10¹¹ CFUs, can be administered to a subject every other day, 3 times per week, 5 times per week, once per month, twice per month, 3 times per month, once every 2 months, or 3 times, 4 times or 6 times per year. In these embodiments, the dose can be administered to the subject for a period extending from about 1 to 2 weeks, 1 to 4 weeks, 1 to 2 months, 1 to 6 months, or 1 to 12 months.

The dose administered to a subject should be sufficient to treat a disease and/or condition, partially reverse a disease and/or condition, fully reverse a disease and/or condition, or establish a healthy-state microbiome. In some aspects, the dose administered to a subject should be sufficient to treat or ameliorate the symptoms of an inflammatory disorder. In some embodiments, the inflammatory is an inflammatory bowel disease such as Crohn's disease or ulcerative colitis.

Suitable doses and dosage regimens can be determined by conventional range-finding techniques known to those of ordinary skill in the art. Generally, treatment is initiated with smaller dosages, which are less than the optimum dose of the active components. Thereafter, the dosage is increased by small increments until the optimum effect under the circumstances is reached. An effective dosage and treatment protocol can be determined by routine and conventional means, starting e.g. with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

Dosing may be in one or a combination of two or more administrations, e.g., daily, bi-daily, weekly, monthly, or otherwise in accordance with the judgment of the clinician or practitioner, taking into account factors such as age, weight, severity of the disease, and the dose administered in each administration.

In another embodiment, an effective amount can be provided in from 1 to 500 ml or from 1 to 500 grams of the bacterial composition having, for example, from 10⁷ to 10¹¹ bacteria per ml or per gram, or a capsule, tablet or suppository having from 1 mg to 1000 mg lyophilized powder having from 10⁷ to 10¹¹ bacteria. Those receiving acute treatment can receive higher doses than those who are receiving chronic administration (such as hospital workers or those admitted into long-term care facilities).

The effective dose as described above, can be administered, for example, orally, rectally, intravenously, via a subcutaneous injection, or transdermally. The effective dose can be provided as a solid or liquid, and can be present in one or more dosage form units (e.g., tablets or capsules).

### Combination Therapies

The compositions described herein comprising a strain of A. hadrus may be combined with other treatment therapies and/or pharmaceutical compositions. For example, a patient suffering from an inflammatory bowel disease, may already be taking a pharmaceutical prescribed by their doctor to treat the condition. In embodiments, the pharmaceutical compositions taught herein, are able to be administered in conjunction with the patient's existing medicines.

For example, the compositions comprising a strain of A. hadrus as described herein may be combined with one or more of: a prebiotic, an anti-diarrheal, a 5-aminosalicylic acid compound, an anti-inflammatory agent, an antibiotic, an antibody (e.g., antibodies targeting an inflammatory cytokine, e.g., antibodies targeting an anti-cytokine agent such as anti-TNF-α, (e.g., adalimumab, certolizumab pegol, golimumab, infliximab, V565) or anti-IL-12/IL-23 (e.g., ustekinumab, risankizumab, brazikumab, ustekinumab), a JAK inhibitor (e.g., tofacitinib, PF0670084 1, PF06651600, filgotinib, upadacitinib), an anti-integrin agent (e.g., vedolizumab, etrolizumab), a SIP inhibitor (e.g., etrasimod, ozanimod, amiselimod), a recombinant cell-based agent (e.g., Cx601), a steroid, a corticosteroid, an immunosuppressant (e.g., azathioprine and mercaptopurine), vitamins, and/or specialized diet.

The compositions, with or without one or more prebiotics, can be administered with other agents in a combination therapy mode, including anti-microbial agents. Administration can be sequential, over a period of hours or days, or simultaneous.

### Diagnostics

In some other embodiments, the level of A. hadrus in a samples (e.g., fecal sample or intestinal biopsy) can be detected using quantitative PCR (qPCR). For example, microbial DNA can be extracted from the sample using methods known to the ordinarily skilled artisan. In qPCR, the 16S rRNA gene from the extracted DNA can be amplified using universal primers and simultaneously quantified using a universal probe. In the same qPCR, a probe specific for the microorganism(s) of interest can be included to quantitate the level of that microorganism(s). For example, a qPCR can include universal primers and a universal probe for the amplification and quantification of total microbial 16S rRNA gene and one or more probes specific for A. hadrus.

Accordingly, in some embodiments, the method for diagnosing an IBD such as UC or CD in a subject comprises: obtaining a sample from the subject; extracting microbial DNA from the stool sample; amplifying 16S rRNA gene from the extracted DNA; quantifying the level of 16S rRNA gene using qPCR; and diagnosing the subject as having CRC or CRA or is at the risk of developing UC or CD when the level of A. hadrus in the sample is increased relative to a control sample.

The following examples are intended to illustrate, but not limit, the disclosure.

### EXAMPLES

### Example 1

This example demonstrates analysis of microbiota from healthy versus diseased subjects to identify the significantly increased prevalence of A. hadrus in healthy subjects as compared to subjects having an IBD.

### A. Methods

### Study Design and Sampling

The study and sample collection were approved by the Clinical Research Ethics Committee of the University College Cork Teaching Hospitals. Informed consent was provided by all involved individuals before undergoing gastrointestinal tract examination at the Cork University Hospital or Bon Secours Hospital in Cork, Ireland. The diagnosis of CD or UC and inflammation status were based on a macroscopic inspection of a gastrointestinal tract during a standard clinical colonoscopy. Adults (>=18 years) undergoing routine monitoring and not diagnosed with any digestive disease or syndrome were regarded as healthy controls. Biopsy samples were obtained from inflamed tissues of adult IBD patients or healthy tissues of control subjects. Exclusion criteria was the use of antibiotics one month prior or during the GI investigation. At the hospitals, the biopsies were collected in 5 mL polypropylene tubes (Sarstedt, Numbrecht, Germany) containing 3 mL RNA-later (Qiagen, Hilden, Germany). The biopsy samples were stored at 4° C. for 24 h before being transferred at −80° C. The associated demographic details and colon location are summarized in Table 1.

### Nucleic Acid Extraction and Sequencing

Biopsies were completely defrosted in RNA-later before performing DNA/RNA purification with AllPrep DNA/RNA/Protein Mini kit (Qiagen). Defrosted biopsies were transferred into a tube containing 350 μL RLT buffer with β-mercaptoethanol (Sigma-Aldrich, St Louis, Mo., USA), three 3.5 mm glass beads and 0.25 mL of 0.1 mm glass beads (Biospec, Bartlesville, Okla., USA). Disruption and homogenization was carried out in a MagNA Lyser (Roche, Penzberg, Germany) twice for 15 seconds at 3,500 or 6,500 rpm. Permutational Multivariate Analysis of Variance (PERMANOVA) test confirmed the different centrifugation speeds did not significantly affect microbiota (data not shown). Subsequent DNA/RNA purification was performed according to the kit manufacturer's instructions. DNA contaminations in RNA samples were removed by Turbo DNA-free kit following manufacturer's instructions (Ambion, Carlsbad, Calif., USA). DNA and RNA concentrations were measured using a Nano-Drop 2000 Spectrophotometer (Thermo Scientific, Waltham, Mass., USA). DNA and RNA integrity were checked on 1% agarose gel electrophoresis and 2100 Bioanalyzer system (Agilent Technologies), respectively. Nucleic acid extracts were stored at −80° C. until further downstream applications. For RNA-16S analysis, total RNA was reverse transcribed using High Capacity cDNA Reverse Transcription kit following manufacturer's instructions (Applied Biosystems, Foster City, Calif., USA). The PCR was employed to amplify 16S rRNA V3-V4 hypervariable region using 341F and 805R primer set with Nextera transposase adaptors (Klindworth et al. 2013, Nucleic Acids. Res. 41(1): e1): 16S_V3_341F, TCGTCGGCAGCGTC_AGATGTGTATAAGAGACAG_CCTACGGGNGGCWGCAG (SEQ ID NO:8); 16S_V4_805R, GTCTCGTGGGCTCGG_AGATGTGTATAAGAGACAG_GACTACHVGGGTATCTAATCC (SEQ ID NO:9). Template DNA or cDNA was mixed with primers at a concentration of 0.2 μM and Phusion High-Fidelity DNA Polymerase for a total volume of 30 μL (Thermo Scientific). PCR conditions were 98° C. for 30 sec, 30 cycles of 98° C. for 10 sec, 55° C. for 15 sec and 72° C. for 20 sec, with final elongation at 72° C. for 5 minutes. PCR products were verified with a presence of a band on an agarose gel and purified using Agencourt AMPure XP magnetic beads (Beckman-Coulter, Brea, Calif., USA). Purified DNA product was eluted in 50 μL EB buffer (Qiagen). Using 5 μL of the PCR products as template, eight additional cycles of PCR was conducted with Illumina primers containing Nextera XT indexes and Phusion High-Fidelity DNA Polymerase in a final volume of 50 μL, then purified using Agencourt AMPure XP magnetic beads. The amplicon concentrations were measured using Quant-iT Picogreen dsDNA assay kit (Thermo Scientific). Libraries were pooled equimolar and sequenced by Illumina MiSeq for 2×300 bp reads at Eurofins Genomics (location). Aliquots of RNA samples were used for host transcriptome RNAseq carried out by Macrogen (location) using TruSeq Stranded mRNA Sample Prep Kit (Illumina) with Illumina HiSeq 4000 2×100 reads following manufacture's protocol.

### Sequencing Data Processing

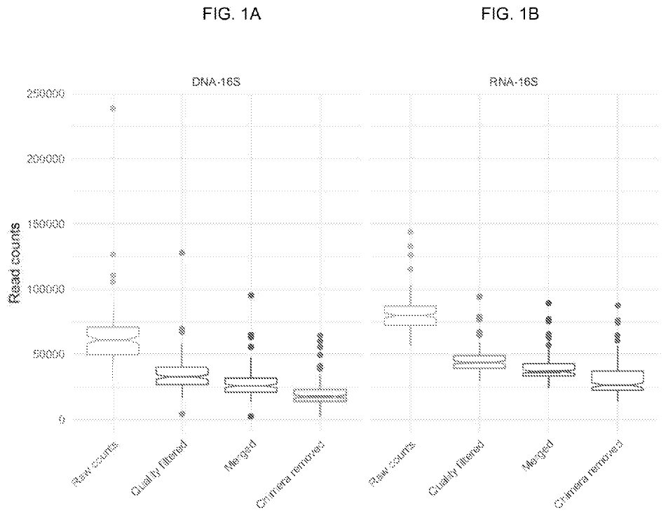

DADA2 (Callahan et al. 2016, Nature Methods, 13:581-583) was used to denoise DNA-16S and RNA-16S sequences. The default parameters were used except the following: for filterAndTrim, trimLeft=c(17, 21), maxEE=c(2, 2), truncLen=c(265, 215) and c(260, 235), for DNA-16S and RNA-16S, respectively; for mergePairs, minoverlap=10 and 20, for DNA-16S and RNA-16S, respectively. Sequence counts distribution at each step is depicted in FIG. 1. After chimera removal, strain level taxonomy was assigned to Ribosomal Sequence Variants (RSVs) using StrainSelect 2016 (world wide web at secondgenome.com/strainselect). In case strain level assignment was not determined, higher level taxonomy was assigned using assignTaxonomy function in DADA2 with Greengenes v13.8 clustered at 97% identity. To reduce sparsity, low abundantRSVs were prevalence filtered at 5%. Then, to remove non-16S rRNA sequences, only the sequences over 400 bp were kept. Samples with <5000 reads were also removed from downstream analyses. For the host RNAseq data, Trimmomatic v0.36 with parameter, 1:30:10 LEADING:20 TRAILING:20 SLIDINGWINDOW:4:20 MINLEN:35, was used to trim Illumina adapters and low quality sequences. Quality filtered reads were aligned to the human genome (GRCh38) using HiSat2 v2. 1.0 (Kim et al., 2015, Nature Methods, 12:357-360) and a count table was generated using featureCounts function in SUBREAD v1.5.2 (Liao et al., 2013, Nucleic Acids Res, 41: e108) using default parameters.

### Statistical Analyses

Data analyses and visualizations were conducted in R statistical environment (R core development team). Microbiota alpha and beta diversities were calculated using phyloseq package (McMurdie and Holmes, 2012, Pac Symp Biocomput, 2012:235-246). PERMANOVA was calculated using adonis function in vegan package (Anderson, 2001, Austral Ecol, 26:32-46). Significantly different microbiota RSVs were determined by zero inflated log-normalized FitFeatureModel in metagenomeSeq package (Paulson et al. 2013, metagenomeSeq: Statistical analysis for sparse high-throughput sequencing. Bioconductor package: 1.11.10 ed. 2013). Since the FitFeatureModel is not able to estimate the model where all samples in one group are zero sequence counts, one count was added to the two deepest sequenced samples if all samples in that group had zero sequence counts prior to the model estimation. Calculated p-values were adjusted by Benjamini-Hochberg method. RSVs with adjusted p-value<0.05, absolute log 2 fold change (abs-log 2FC)>1 and non-zero sequence counts in at least 75% of the subjects in at least one group (CD, UC or Control) were defined as dynamic RSVs. Differential expression analysis on host RNAseq was performed using DESeq2 package (Love et al., 2014, Genome Biol, 15:550) with betaPrior=TRUE option for DESeq function. Pathway enrichment analysis was carried out using the ReactomePA package (Yu and He, 2016, Molecular BioSystems).

### B. Results

### Cohort Demographics

The study cohort consisted from the patients with IBD, who had been diagnosed for 10 years on average, and were receiving IBD medications for acute symptoms (Table 1).

TABLE 1
Study cohort demographics

| | CD | UC | Control | |
|---|---|---|---|---|
| No. of total | 58 | 88 | 39 | |
| subjects | | | | |
| DNA-16S | 42 | 61 | 29 | |
| RNA-16S | 46 | 75 | 25 | |
| HostRNAseq | 52 | 83 | 27 | |
| Gender | 45 | 48 | 41 | Chi-Square |
| (% Female) | | | | p-0.74 |
| Age (y) | 41.6 ± 12.3 | 47.7 ± 13.0 | 58.4 ± 11.6 | ANOVA |
| Mean ± SD | | | | p < 0.001 |
| Colon location | | | | |
| Ascending | 4 | 10 | 3 | |
| Cecum | 1 | 13 | 1 | |
| Descending | 3 | 2 | 7 | |
| Rectum | 2 | 1 | 11 | |
| Sigmoid | 5 | 1 | 12 | |
| Transverse | 3 | 1 | 0 | |
| Diagnosis period | 11.3 ± 8.68 | 10.7 ± 8.14 | NA | ANOVA |
| (y) Mean ± SD | | | | p = 0.67 |

This cohort included 185 biopsy samples from inflamed colonic mucosa acquired from 58 CD, 88 UC and 39 control subjects. Age was significantly higher among the controls (Chi-square test p<0.001), which was expected as older people are more likely to undertake routine colonoscopy and histology. Gender was, however, not significantly different across the three groups (ANOVA p=0.743). To understand whether any specific microbiota was correlated with age, a correlation test was carried out between age and DNA-16S or RNA-16S RSV abundance. The highest correlation coefficients were rho=0.29 and 0.3 for DNA-16S and RNA-16S RSVs, respectively, and none of them were significant (adjusted p>0.05). With these results, following statistical tests were not adjusted with the age.

Microbiota Composition and Diversity of Both DNA-16S and RNA-16S are Associated with Disease

To profile the microbiota of the intestinal mucosa in UC, CD and controls, 16S rRNA gene abundance and expression were examined by sequencing DNA (DNA-16S) as well as cDNA (RNA-16S) for available samples: DNA-16S, 61 UC, 42 CD, and 29 controls; RNA-16S, 75 UC, 46 CD and 25 control samples; 35 CD, 52 UC and 15 controls are in common between DNA-16S and RNA-16S. After all pre-processings described in methods, the de-noised RSV counts were 14,872±6,048 and 21,324±7,742 (mean±SD), for DNA-16S and RNA-16S, respectively. Sequences were de-noised to result in 313 and 435 RSVs, for DNA-16S and RNA-16S, respectively. PERMANOVA test confirmed that RSV composition was significantly different across the CD, UC and control groups in DNA-16S (p=0.002) as well as RNA-16S (p=0.001). In addition, RSV composition also differed significantly in the age groups (p=0.021) in DNA-16S but not in RNA-16S. Gender was not associated with RSV compositions in both dataset (p>0.05). Alpha diversity was significantly different between CD, UC and control groups by Kruskal-Wallis test in both datasets (p<0.05; FIG. 2A (DNA-16S richness and Shannon diversity) and FIG. 2B (RNA-16S richness and Shannon diversity). Wilcoxon rank sum test was used to further compare the differences between groups. All combinations except Shannon diversity between UC versus Controls demonstrated significant differences (p<0.05) in both datasets.

Microbial Composition of RNA-16S Samples Demonstrated a Shift from DNA-16S Samples

DNA-16S and RNA-16S data were compared using 102 samples which have both datasets. From the total of 486 RSVs, 262 were shared between two datasets, and 51 and 173 RSVs were unique to DNA-16S and RNA-16S, respectively. Principal Coordinate Analysis (PCoA) on Bray-Curtis dissimilarity matrix indicated a marked shift from DNA-16S (circles) to RNA-16S (triangles) (FIG. 3), with the PERMANOVA test confirming significant difference between two datasets (p=0.001). Ellipses demonstrated similar shift by disease status (CD, UC or control) between DNA-16S and RNA-16S.

### Anaerostipes Hadrus Metabolic Activity was Significantly Depleted in Both UC and CD

Microbial features associated with CD and UC compared to controls in DNA-16S and RNA-16S were then identified. Three and twelve RSVs were significantly (adjusted p<0.05 and abs-log 2FC>1) depleted in CD and UC compared to Controls, respectively, in DNA-16S dataset (FIG. 4). Among those, solely Coprococcus RSV2 was the dynamic RSV (see definition in Example 1 Methods section above) depleted both in CD and UC. Three additional RSVs were dynamic and depleted in UC samples in DNA-16S; Anaerostipes hadrus RSV and two other RSVs specific to a single strain, Coprococcus catus GD/7 and Gemmiger formicilis X2-56. In the RNA-16S dataset, 10 and 9 RSVs were significantly (adjusted p<0.05 and abs-log 2FC>1) depleted in CD and UC compared to controls, respectively (FIG. 4). Of those, a total of 10 RSVs were dynamic, including A. hadrus RSV (depleted in both CD and UC), C. catus GD/7 (depleted in CD), G. formicilis X2-56 (depleted in UC), Slackia isoflavoniconvertens HE8 (depleted in UC), Sutterella wadsworthensis 2_1_59BFAA (enriched in CD), Turicibacter sanguinis MOL361 (depleted in CD), unclassified Coprococcus RSV1 (enriched in CD) and RSV2 (depleted in UC), unclassified Lachnospiraceae family RSV1 (depleted in UC) and RSV2 (depleted in CD).

### Host RNAseq Confirmed Inflammation in CD and UC Compared to Controls

The quality-trimmed host RNAseq reads from RNAseq library of 162 samples (52 CD, 83 UC and 27 controls) were aligned to 32,471 genes with the total read counts 19,590,032±7,012,749 (mean±SD) per sample. PERANOVA test using Euclidean distance between samples demonstrated that the CD, UC and control groups were significantly different (p=0.001), which is illustrated as a shift of the CD, UC away from control groups in a principal component analysis (data not shown). Moreover, the age (p=0.008) but not gender (p=0.096) was significantly associated with host gene expression by PERMANOVA tests. Of the 1,579 genes were significantly differentially expressed in CD compared to controls (adjusted p<0.05 and abs-log 2FC>1). Among those genes, FOLH1 (log 2FC=4.02), MTTP (log 2FC=3.71) and CXCL8 (log 2FC=3.68) exhibited the highest abs-log 2FC and were all upregulated in CD. CXCL8 encodes for a neutrophil chemokine, a major mediator of the inflammatory response and was shown to be elevated in the intestinal mucosa of IBD patients with the active disease state (Mazzucchelli et al., 1994, Am J Pathol, 144:997-1007). FOLH1 encodes a folate hydrolase that was found to be elevated in IBD patients by Rais et al. (2016, JCI Insight, 1: e88634). Of the 1,469 genes significantly differentially expressed in the UC compared to controls, C2CD4A (log 2FC=3.50), DUOXA2 (log 2FC=3.49) and IGHGP (log 2FC=3.37) showed the highest gene upregulation in UC. C2CD4A is known to be involved in inflammatory response (Warton et al., 2004, Gene, 342:85-95). Pathway enrichment analysis demonstrated significant change in IL-4/IL-13, extracellular matrix organization, collagen degradation, and IL-10 signaling pathways in both CD and UC patients compared to controls. These results confirmed elevated inflammatory response and mucosal damages in CD and UC patients compared to controls.

A. hadrus Metabolic Activity was Associated with Termination of O-Glycan Biosynthesis Pathway

Since the A. hadrus RSV was the only one dynamically depleted in both UC and CD compared to controls in RNA-16S datasets, further investigation was done to determine whether its activity was associated with host gene expressions by comparing A hadrus RSV RNA-16S abundance against host RNAseq irrespective of host disease status. As a result, 191 significantly differentially expressed genes (adjusted p-value<0.05) were identified which are associated with the A. hadrus RSV, of which 115 genes were not differentially expressed between either UC or CD compared to controls. Pathway enrichment analysis of the 191 genes identified 17 significantly enriched pathways (adjusted p<0.05). Each pathway was investigated to find a series of significantly differentially expressed genes. As a result, termination of O-glycan biosynthesis pathway (adjusted p=0.002) was identified, which included MUC12, MUC13, ST3GAL4 and ST6GALNAC4 genes that were significantly negatively and MUC6 that was significantly positively associated with A. hadrus.

### Example 2

Use of A. hadrus to Treat an Intestinal Inflammatory Disorder

This example studies the effects of administering live bacteria on glucose homeostasis by performing glucose tolerance tests (GTTs) on the high-fat diet-induced obese mouse model. The mice prepared and used as described in WO2019067087, which incorporated by reference in its entirety herein.

IBD is induced in mice by supplementing drinking water with 3% dextran sodium sulfate for 7 days. For each daily treatment, an A. hadrus liquid culture is grown to an optical density of 0.4-0.5 and then pelleted by centrifugation. Bacteria are resuspended in phosphate buffered saline and 100 μl is administered by oral gavage to mice beginning on Day 8. Mice are treated daily for 1 week with the A. hadrus suspension or with phosphate buffered saline alone as a negative control. After 5 days of bacterial treatment, colitis is scored in live mice using endoscopy. Endoscopic damage score is determined by assessing colon translucency, fibrin attachment, mucosal and vascular pathology, and/or stool characteristics. Mice are sacrificed after 7 days of treatment and colonic tissues are isolated. Distal colonic sections are fixed and scored for inflammation and ulceration. A reduction in endoscopic damage observed after 5 days of treatment and/or a reduction in inflammation and/or ulceration of distal colonic sections from sacrificed mice is indicative of a therapeutic effect of the A. hadrus treatment.

Although the foregoing invention has been described in some detail by way of illustration and examples, which are for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

1. A method for treating a subject in need thereof, the method comprising administering to the subject a composition comprising a therapeutically effective amount of a bacterium, wherein the bacterium has a 16S RNA gene that is at least 90%, 91%, 92%, 93%, 94%, 95%, 96%, 97%, 98%, or 99% identical to SEQ ID NO:8.

2. The method according to claim 1, wherein the bacterium has a 16S RNA gene that is at least 99.1%, 99.2%, 99.3%, 99.4%, 99.5%, 99.6%, 99.7%, 99.8% or 99.9% identical to SEQ ID NO:8.

3. The method according to claim 1, wherein the bacterium administered to the subject is viable.

4. The method according to claim 1, wherein the subject has been diagnosed with a gastrointestinal disorder.

5. The method according to claim 4, wherein the gastrointestinal disorder is selected from the group consisting of ulcerative colitis, Crohn's disease, inflammatory bowel disease, irritable bowel syndrome, and celiac disease.

6. The method according to claim 1, wherein the therapeutically effective amount of the bacterium comprises about 1×10⁷ to 1×10¹² colony forming units (CFU) of the bacterium.

7. The method according to claim 1, wherein the bacterium is A. Hadrus.

8. The method according to claim 1, wherein the method comprises administering the composition to the subject once, twice or three times per day over a time period of about 1-52 weeks.

9. The method according to claim 1, wherein the weight loss of the subject after administration of the composition is less than that of a comparable patient who did not receive administration of the composition.

10. The method according to claim 9, wherein the weight loss of the subject after administration of the composition is about 5 lbs, 10 lbs, 15 lbs, 20 lbs, 25 lbs or 30 lbs less than that of a comparable patient who did not receive administration of the composition.

11. The method according to claim 10, wherein the weight loss of the subject is measured at 3 months, 4 months, 5 months, 6 months, 7 months, 8 months, 9 months, 10 months, 11 months, or 12 months after the first administration of the composition.

12. A method for maintaining or improving overall fitness or health of a patient, said method comprising:
administering to the patient a therapeutic composition comprising:
i. an effective amount of a composition comprising a bacterium, wherein the bacterium has a 16S RNA gene that is at least 90%, 91%, 92%, 93%, 94%, 95%, 96%, 97%, 98%, or 99% identical to SEQ ID NO:8; and
ii. a pharmaceutically acceptable carrier.

13. The method of claim 12, wherein the overall fitness or health of the patient after administration of the composition is greater than that of a comparable patient who did not receive administration of the composition.


SEQUENCE LISTING (9 sequences)

SEQ ID NO: 1 - DNA, 1434 bp, Anaerostipes hadrus
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaag

SEQ ID NO: 2 - DNA, 1521 bp, Anaerostipes hadrus
tttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctaccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccttatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggccttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctt

SEQ ID NO: 3 - DNA, 1521 bp, Anaerostipes hadrus
tttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtaggggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctt

SEQ ID NO: 4 - DNA, 1515 bp, Anaerostipes hadrus
tggctcaggatgaacgctggcggcgtgcttaacacatgcaagtcgaacgaagctgcttaactgatcttcttcggaattgacgttttgtagactgagtggcggacgggtgagtaacgcgtgggcaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatttcaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctttc

SEQ ID NO: 5 - DNA, 386 bp, Anaerostipes hadrus
gagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagc

SEQ ID NO: 6 - DNA, 1455 bp, Anaerostipes hadrus
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtg

SEQ ID NO: 7 - DNA, 1455 bp, Anaerostipes hadrus; identical to SEQ ID NO: 6

SEQ ID NO: 8 - DNA, 50 bp, Artificial
  Synthetic oligonucleotide
  misc_feature (42)..(42): n is A or T
tcgtcggcagcgtcagatgtgtataagagacagcctacgggnggcwgcag

SEQ ID NO: 9 - DNA, 55 bp, Artificial
  Synthetic oligonucleotide
gtctcgtgggctcggagatgtgtataagagacaggactachvgggtatctaatcc